(19) United States
(12) Patent Application Publication
(10) Pub. No.: US 2004/0229330 A1
(43) Pub. Date: Nov. 18, 2004

(54) Integrated method for capture and purification of tagged proteins

(75) Inventors: Jeffrey David Bettencourt, Walnut Creek, CA (US); David C. Boisvert, El Cerrito, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Bayer Pharmaceuticals Corporation, West Haven, CT

(21) Appl. No.: 10/789,246

(22) Filed: Feb. 27, 2004

Related U.S. Application Data
(60) Provisional application No. 60/451,093, filed on Feb. 27, 2003.
(60) Provisional application No. 60/502,923, filed on Sep. 15, 2003.

(51) Int. Cl. C12N 9/00

(52) U.S. Cl. 435/183; 530/412

(57) ABSTRACT

The present invention provides a method for high efficiency capture and purification of a tagged protein from a protein preparation, especially a tagged protein of low quantity. The method comprises a first concentration step, concentrating a target tagged protein by a negatively charged capture support; and a second purification step, purifying the tagged protein by a tag-specific affinity support. The method is especially useful for capturing and purifying tagged protein of low quantity. The present invention also provides a kit for the capture and purification of the tagged protein.

### CROSS-REFERRENCE

This application claims the benefit of U.S. Provisional application No. 60/451,093, filed Feb. 27, 2003 and 60/502,923, filed Sep. 15, 2003, which are incorporated by reference in their entirety.

### BACKGROUND OF THE INVENTION

The process of moving protein drugs through initial discovery and early proof of principle stages has been facilitated by expression as fusion proteins with purification tags such as polyhistidine and antibody Fc domain. In principle these tags allow target proteins to be retrieved from crude cell culture supernatants with very high final purity levels in a single chromatography step. Polyhistidine tags such as 6×His can be captured and purified on immobilized metal affinity chromatography supports (IMAC) such as nickel nitrilotriacetic acid (Ni-NTA), and Fc fusion proteins can be captured and purified on antibody-binding resins such as immobilized Protein-A or Protein-G, or on any of several synthetic antibody-specific chromatography resins currently marketed, such as MEP-Hypercell® (BioSepra, Cergy, Saint Christopher, France), ABx (JT Baker Chemical Co., Phillipsburg N.J.), antibody-selective resins from ProMetic Biosciences, (Wayne, N.J.), also MabSelect from Amersham Biosciences (Piscataway, N.J.). Because the chromatographies are specific to the tag and not the attached target protein, these methods are generic, and resources do not have to be expended defining new chromatography systems for each new target research protein molecule.

In practice the low protein expression levels often seen in transient mammalian cell culture systems frustrate attempts to use these tag capture schemes effectively. At low concentrations of 1-2 mg/liter or less, binding of tagged target molecules to affinity matrixes like Protein-A/G or Ni-NTA is often thermodynamically unfavorable, and less than 25% of the target protein may be bound out of culture supernatant. Capture of polyhistidine-tagged proteins by IMAC matrixes is also complicated by the presence of small molecules in the cell culture medium, such as histidine and cysteine, that compete for binding to the IMAC support, interfering with capture.

These problems can be overcome if the cell culture supernatant is concentrated by some means, and interfering small molecules washed away from the system. Typically this has been done by membrane concentration (also called ultrafiltration) followed by buffer exchange (also called diafiltration). Ultrafiltration devices utilize controlled pore size membranes that allow water and small molecules such as amino acids to pass freely to waste, while retaining larger molecules like proteins. The initial concentration operation increases the concentration of the tagged protein to the point where binding to the appropriate affinity matrix is more thermodynamically favorable, and the diafiltration step washes away small molecules that would otherwise out-compete the tagged molecule for binding. However, ultrafiltration requires specialized equipment that differs radically as the scale of the operation increases, and which may not be available to all investigators. Preparation, operation, and sanitization of the equipment is also labor intensive, and few automated systems are available.

A chromatography-based concentration system would be an attractive alternative to ultrafiltration and diafiltration. Chromatography is easily scalable and automated equipment is commonplace. If a non-affinity chromatography matrix such as an ion exchanger could be used to bind proteins indiscriminately out of culture supernatants, large concentration factors of several hundred-fold could be obtained, as the protein elution volume would be very small compared to the culture supernatant feed volume. A simple isocratic wash would effectively remove interfering small molecules, and the eluted impure protein mixture could then be applied to the tag-specific affinity chromatography matrix for final purification. In practice this is often impractical because the ionic strength of mammalian cell culture media is high enough that significant dilution is required to allow binding to ion exchangers to occur. Not only does this become less practical as scale increases and demand for diluent expands, but further dilution of the target molecule may make efficient capture on the ion exchanger impossible.

There is yet another problem that may make capture and purification of Fc-tagged proteins impractical. The standard condition for elution of Protein-A/G resins, as well as other synthetic antibody-specific affinity matrixes (such as MEP-Hypercell®, ABx, ProMetic antibody-selective resins, and MabSelect), is reduction of mobile phase pH to the range of 2-4. Acidic pH values in this range often lead to aggregation of the tagged target molecule, and loss of biological activity. Non-denaturing neutral pH conditions for elution of these matrixes would make the use of Fc-tagged fusion expression more practical and predictable.

### SUMMARY OF THE INVENTION

The invention relates to methods and kits for high efficiency capture and purification of a tagged protein. The current invention is particularly useful if the tagged protein is present at low quantity.

In one aspect, the invention provides a method for purification of a tagged protein from a protein preparation comprising a concentration step and an affinity purification step. The concentration step further comprises steps of: contacting the protein preparation with a capture support; washing the capture support with a capture support washing buffer of low ionic strength to remove interfering molecules but not the tagged protein from the capture support; and eluting the tagged protein from the capture support with a capture support eluting buffer of high ionic strength. The affinity purification step further comprises the steps of: contacting the capture support eluate with a tag-specific affinity support; washing the affinity support with affinity support washing buffer of low ionic strength to remove some impurities but not the tagged protein from the affinity support; and eluting the tagged protein from the affinity support with an affinity support eluting buffer.

In one embodiment of the invention, the negatively charged capture support comprises a polyanion, such as heparin.

In another embodiment of the invention, the tagged protein is a polyhistidine tagged protein, and the affinity support comprises nickel nitrilotriacetic acid. The polyhistidine-tagged protein is eluted from the affinity support with a eluting buffer comprising at least 50 mM imidazole.

In yet another embodiment of the invention, the tagged protein is an Fc-tagged protein, and the affinity support comprises protein A and/or protein G. The Fc-tagged protein is eluted from the affinity support with a non-denaturing buffer with a neutral pH.

In another aspect of the invention, a kit for the capture and purification of a tagged protein is provided, wherein the kit in separate containers comprises a negatively charged capture support, and a tag-specific affinity support.

Among the several advantages of the present invention it may be noted the provision of an efficient method of capture and purification of a tagged-protein of low quantity by which the tagged-protein is first concentrated using a negatively charged capture support, before proceeding to the purification of the tagged protein using a tag-specific affinity support.

### OBJECTS OF THE INVENTION

Accordingly, it is an object of the present invention to provide a method to facilitate high efficiency capture of tagged molecules from culture supernatant.

It is another object of the present invention to provide a method to facilitate the high efficiency capture of histidine tagged molecules from culture supernatant.

It is also an object of the present invention to provide an improved method of purifying low quantities of histidine tagged molecules.

It is a further object of the present invention to provide an improved method of purifying Fc fusion proteins to a high level of purity by means of non-denaturing, neutral pH elution conditions.

It is yet another object of the present invention to provide a kit for the capture and purification of a tagged protein from a protein preparation.

These and other objects of the invention will become apparent in light of the detailed description below.

### DESCRIPTION OF THE FIGURES

FIG. 1 presents an idealized binding isotherm illustrating the difficulty frequently encountered in capturing tagged targets, such as poly-His fusions on IMAC matrixes, from low-expression cell culture supernatants.

FIG. 2 illustrates a schematic diagram of an immobilized heparin chromatography resin supporting its utility as a generic concentration medium.

FIG. 3 shows the result of loading Millipore ProsepTM Heparin with unadjusted cell culture supernatant, to a target of 10 mg/ml total protein. Lane 1 is cell culture supernatant; lane 2 represents heparin flowthrough, 5% of load; lane 3 represents heparin flowthrough, 38% of load; lane 4 represents heparin flowthrough, 71% of load; line 5 represents heparin flowthrough, 100% of load; lane 6 represents heparin, 500 mM NaCl eluate; and lane 7 represents heparin, 2 M NaCl.

FIG. 4 illustrates a schematic of the mercaptoethyl pyridine ligand of MEP Hypercell®.

FIG. 5 illustrates elusion of Fc-fusion target from MEP Hypercell and Protein-A screen of non-acidic elution conditions. Condition no. 1: Pierce Ab-proetin-A elustion buffer; Condition no. 2: 100 mM CAPS, pH 10.4; condition no. 3: 100 mM CAPS, 50% ethyl glycol, pH 10.5; and condition no. 4: 100 mM Tris, 3.5 M MaCl2, 0.1% Tween 80, pH 8. MEP Hypercell base case buffer is 100 mM sodium acetate, pH 4.0. Protein-A base case buffer is 200 mM glycine, pH 3.0.

FIG. 6 illustrates elusion yields of Fc-fusion target from MEP Hypercell as a function of pH dependence using an 50% ethylene glycol buffer system. All test buffers were 100 mM Tris, 100 mM glycine, and 50% w/v ethylene glycol.

FIG. 7 shows a gel and blot from experiment No. 3 where the following materials where loaded: cell culture supernatant containing an Fc-tagged target cytokine at ˜1 mg/liter (lane 1); cell culture supernatant pre-concentrated by immobilized heparin chromatography (heparin concentration eluate) (lane 2); heparin concentration eluate applied to MEP Hypercell (MEP Hypercell eluate)(lane 3); and cell culture supernatant applied directly to a Protein-A Hyper-DTM column, eluted with 3.5 Molar MgCl2, pH 7.5 (lane 4).

### DETAILED DESCRIPTION OF THE INVENTION

The present invention relates to a set of integrated tools for efficient capture of tagged molecules from a protein preparation from a source such as cell lysate or culture supernatant, and subsequent polishing to high purity. The system features a generic “capture support” utilizing immobilized negatively charged polyanions, including polyanionic polysaccharides, such as heparin that concentrates the target and removes interfering small molecules without prior adjustment of the protein preparation. The system further provides a tag-specific affinity support to purify the tagged protein to high purity.

One embodiment of the invention provides a method for capture and purification of a tagged protein from a protein preparation comprising a concentration step and an affinity purification step. The concentration step further comprises steps of: contacting the protein preparation with a capture support; washing the capture support with a capture support washing buffer of low ionic strength to remove interfering molecules but not the tagged protein from the capture support; and eluting the tagged protein from the capture support with a capture support eluting buffer of high ionic strength. The affinity purification step further comprises the steps of: contacting the capture support eluate with a tag-specific affinity support; washing the affinity support with affinity support washing buffer of low ionic strength to remove some impurities but not the tagged protein from the affinity support; and eluting the tagged protein from the affinity support with an affinity support eluting buffer.

As used herein the term “low ionic strength” refers to a buffer containing a salt concentration of no more than 50 mM, preferably no more than 150 mM. For example, for a heparin concentration support, a low ionic strength wash can be a phosphate or similar buffer of around 50 mM, with no additional salt, and a wide range of salt concentrations can be used in a wash step, such as 150 mM to 2 Molar, since affinity supports are relatively insensitive to salt.

As used herein the term “high ionic strength” refers to a buffer containing a salt concentration of at least 400 mM, preferably at least 500 mM.

Affinity supports are relatively salt-insensitive and are eluted by more specific buffer conditions. For example, His-tagged targets can be eluted from an IMAC support by inclusion of imidazole, a specific eluent, in the elution buffer; Fc-tagged targets on MEP-Hypercell can be eluted by 50% ethylene glycol at neutral or basic pH; and Fc-tagged targets on Protein-A can be eluted with a high concentration of MgCl₂, a chaotropic salt, at a concentration of 3-4 Molar.

As used here the term “interfering molecules” refers to impurities and small molecules in the protein preparation that may interfere with purification of target tagged protein by the affinity support, including, but not limited to, histidine, and cysteine.

The contacting of a protein preparation with the negatively charged capture support, and the subsequent washing and eluting steps can be performed in a column, a beaker, a flask, a test tube, or equivalent thereof.

Similarly, the contacting of the eluate from the capture support with the tag-specific affinity support, and the subsequent washing and eluting steps can be performed in a column, a beaker, a flask, a test tube, or equivalent thereof.

In one embodiment of the invention, the negatively charged capture support comprises heparin. Heparin is a naturally occurring polysaccharide that is highly substituted with carboxyl and sulfate groups, giving the molecule an extremely high density of negative charge at physiological pH. This charge density allows heparin immobilized on a chromatography support to bind proteins indiscriminately out of cell culture supernatants via the positively charged surface amino acids lysine, arginine, and histidine. Prior dilution or adjustment of feed pH is generally not required. A wash of low ionic strength removes small molecules that might interfere with IMAC purification, and an elution with sodium chloride at approximately 0.5 molar elutes an impure protein mixture highly concentrated relative to the initial feed volume. The tagged target protein in this mixture can then be polished to a high degree of purity by affinity purification, either by IMAC chromatography in the case of polyhistidine-tagged molecules, or by Protein-A/G or antibody-specific affinity matrixes in the case of Fc-tagged molecules. Non-limiting examples of affinity matrixes include MEP-Hypercell® (BioSepra, Cergy, Saint Christopher, France), ABx (JT Baker Chemical Co., Phillipsburg N.J.), antibody-selective resins from ProMetic Biosciences, (Wayne, N.J.), also MabSelect from Amersham Biosciences (Piscataway, N.J.). Binding to heparin is not necessarily restricted to binding on the tag itself, but may occur through interaction with basic residues on the surface of the target protein bearing the tag, specifically histidine, arginine, and lysine residues. Thus, it is important to note that while our development of the heparin concentration step has so far been restricted to Fc and polyhistidine tagged proteins, it is generally applicable to the problem of efficient capture of molecules with other types of purification tags, such as the GST and Chitin Binding Domain tags.

Other polyanionic polysaccharides suitable for the current invention include, but not limited to, hyaluronic acid (HA), demmatan sulfate, carboxymethylcellulose (CMC), carboxymethylamylose (CMA), chondroitin-6-sulfate, dermatan sulfate, hyaluronic acid, alginic acid, polyuronic acid, and other negatively charged glycosaminoglycans.

As used herein the term “glycosaminoglycan” includes reference to a polysaccharide composed of repeating disaccharide units. The disaccharides always contain an amino sugar (i.e., glucosamine or galactosamine) and one other monosaccharide, which may be an uronic acid (i.e., glucuronic acid or iduronic acid) as in hyaluronic acid, heparin, heparin sulfate, chondroitin sulfate or dermatan sulfate—or galactose as in keratan sulfate. The glycosaminoglycan chain may be sulfated on either moiety of the repeating disaccharide.

“Polyanion,” as used herein, refers to a molecule that possesses a plurality of negative charges. “Polyanionic polysaccharide,” as used herein, includes reference to carbohydrates that possess a plurality of negative charges. As used herein the term “heparin” refers to a naturally occurring, or synthetic linear glycosatninoglycan consisting of an alternating 1-4-linked glucosamine (GlcN) and hexuronic acid (HexA) residues. This simple repeat structure acquires a considerable degree of variability by extensive modifications involving sulfations and uronate epimerization. Amino groups of the GlcN units are either sulfated or acetylated, and the types of uronic acid residues are either iduronate or glucuronate. O-Sulfate substitutions occur at C6 or the GlcN and at C2 or the uronic acid residues. Combination of all these modifications gives rise to very diverse structures. Heparin exists in a wide range of molecular weights from 5,000-40,000. Minute amounts of other sugars may also be present. Heparin is highly charged and strongly acidic.

“Dermatan Sulfate” (DS), as used herein, includes reference to a heterogeneous glycosaminoglycan mixture that contains disaccharide repeat units consisting of N-acetyl-D-galactosamine and D-glucuronic acids, as well as disaccharide repeat units consisting of N-acetyl-D-galactosamine and L-iduronic acid. The N-acetyl-D-galactosamine residues may be sulfated on the 4 and/or the 6 position. The uronic acids are present with variable degrees of sulfation.

“Chondroitin sulfate” (CS), as used herein, includes reference to a heterogeneous glycosaminoglycan mixture that contains disaccharide repeat units consisting of N-acetyl-D-galactosamine and D-glucuronic acids. The N-acetyl-D-galactosamine residues may be sulfated on the 4 and/or the 6 position.

“Hyaluronic acid”, as used herein, includes reference to a heterogeneous glycosaminoglycan mixture that contains disaccharide repeat units consisting of N-acetyl-D-glucosamine and D-glucuronic acids.

“Alginic acid”, as used herein, includes reference to a heterogeneous polysaccharide mixture that contains L-glucuronic acids and manuronic acids As used herein, the term “protein preparation” refers to a source of target tagged protein obtained from, but not limited to, cell lysate, culture supernatant, tissue sample, or protein in vitro synthesized in rabbit reticulocyte lysate.

In one embodiment of the invention, the tagged-protein is a polyhistidine tagged protein, preferably a 6× histidine tagged-protein. Polyhistidine-tagged molecules can be polished to high purity using either copper, nickel, or zinc metal ions immobilized on IDA (iminodiacetic acid), NTA (nickel nitrilotriacetic acid), or TED (tricarboxyethylenediamine) IMAC ligands. The most frequently encountered combination would be the hexahistidine tag, 6×His, purified on Ni-NTA resin. The Ni-NTA system is highly specific for the 6×His tag, and final purities of greater than 95% are typical.

In another embodiment of the invention, the tagged-protein is an Fc-tagged protein. The conventional method to elute Fc-tagged protein from the Fc-binding ligand is by lowering pH. Acidic pH values, however, often lead to aggregation of the tagged-proteins, and loss of biological activity. The difficulty of acid-induced aggregation and inactivation of Fc-tagged target proteins is overcome in our system by the use of 4 Molar magnesium chloride at or near neutral pH. The Fc tag affinity binding interaction, either with Protein-A/G or with an antibody-specific affinity matrix, is a combination of ionic, hydrophobic, and other weak intermolecular interactions that in total produce a very strong binding interaction. Disrupting at least some of these interactions can potentially elute bound Fc from an affinity matrix. The classical acid pH elution scheme used for these matrixes causes a partial denaturation, or unwinding, of one or both of the binding partners. Sites of mutual attraction between the affinity ligand and the Fc no longer line up in a manner that favors binding, and the Fc-tagged protein elutes. Unfortunately, the partially denatured Fc-tagged protein may be subjected to aggregation and inactivation in the process.

In one embodiment of the invention, Fc-tagged protein is eluted from the affinity support with an affinity support eluting buffer comprising MgCl₂ at a concentration of at least 3 molar, preferably at least 4 molar. Magnesium chloride is a salt of intermediate chaotropic strength, and is capable of disrupting hydrophobic interactions between bound molecules. In our system this high chaotropic potential, in combination with the very high ionic strength of the 4 molar magnesium chloride, probably disrupts hydrophobic and ionic interactions between the Fc tag and the Protein-A/G affinity ligand, allowing the tagged protein to desorb at neutral pH without denaturation.

An alternative antibody-specific affinity matrix, such as MEP Hypercell, that shows strong affinity to antibodies independent of subclass and species is particularly preferred for purifying Fc-tagged protein. Such a matrix allows for the development of a generic method for purification of any Fc-tagged protein regardless the species and subclasses of the Fc tag.

In another embodiment of the invention, the Fc-tagged protein is purified by other antibody-specific affinity support such as MEP Hypercell, and the Fc-tagged protein is eluted by a eluting buffer at neutral pH, such as 50% ethylene glycol, which is a solvent well known for its ability to disrupt hydrophobic interactions. The MEP (mercaptoethyl pyridine) ligand presents three functionalities: a pyridine ring provides hydrophobic interaction, a short ethyl spacer provides some additional hydrophobic interaction, and a thioether group gives the ligand some additional preference for antibodies or Fc fragments. The spacing of these functionalities along the ligand is apparently also optimized for binding to antibodies and Fc fragments.

Other antibody-specific affinity supports with similar properties are suitable for use in the current invention for the purification of an Fc-tagged protein including, but not limited to, ABx (JT Baker Chemical Co., Phillipsburg N.J.), antibody-selective resins from ProMetic Biosciences, (Wayne, N.J.), also MabSelect from Amersham Biosciences (Piscataway, N.J.).

Other non-ionic solvents, including but not limited to, Triton X-100, isopropanol, and acetonitrile are contemplated for use as affinity support eluting buffer to elute Fc-tagged protein from an antibody-specific affinity support.

The protein purification method of the invention comprising a first concentration step and a second purification step also applies to purification of other tagged proteins including but not limited to GST-tagged protein, Myc-tagged protein, hemagglutinin (HA)-tagged protein, Green fluorescent protein (GFP)-tagged protein, and flag-tagged protein. His tags typically bind to IMAC supports such as NiNTA (Nickel nitrilotriacetic acid), while Fc tags typically bind to antibody-specific supports such as Hypercell and Protein-A. One of skill in the art will recognize that a support chosen for the purification step will be specific to the tag used.

The present invention can also be assembled into kits. When the invention is supplied as a kit, the different components of the composition may be packaged in separate containers and admixed or rehydrated prior to use. One embodiment of the invention provides a kit for the purification of tagged protein comprising a negatively charged capture support, and a tag-specific affinity support. In another embodiment of the invention, the kit further comprises one or more of the following: a capture support washing buffer, a capture support eluting buffer, an affinity support washing buffer, an affinity support eluting buffer, and instruction for using the kit.

In one embodiment of the invention, the capture support in the kit comprises heparin.

In another embodiment of the invention, the tagged protein is a polyhistidine-tagged protein, and the affinity support in the kit comprises nickel nitrilotriacetic acid.

In yet another embodiment of the invention, the tagged protein is an Fc-tagged protein, and the affinity support in the kit comprises protein A/G resin.

In one embodiment of the invention, the buffer is supplied either in a concentrated form, or as an anhydrous preparation. Any buffers that maintain suitable pH for the working solution and do not interfere with the binding of tagged-protein with the support are contemplated. The suitable range for the current invention is between about pH 6.0 to about pH 9.0, preferably between about pH 7.0 and about pH 8.0. Suitable, but non-limiting, buffers include HEPES, PBS, PIPES, Tris-Hydrochloride (Tris-HCl), and MOPS.

When the invention is provided in a kit, the different components of the invention may comprise subsets of these parts and may be combined in any way that either facilitates the application of the invention or prolongs storage life.

Methodology

The following components, formulations and procedures may be used in practicing this invention. They are set forth for explanatory purposes only, and are not to be taken as limiting the invention.

Heparin Column Concentration

A column packed with immobilized heparin, such as Toso Haas Heparin 650M, can be packed and equilibrated in phosphate buffered saline, or a similar buffer in the range of pH 7.2 and an ionic strength equivalent to approximately 130 mM NaCl. Cell culture supernatant can then be perfused through the column. The volumetric binding capacity of the heparin column will vary with feed total protein concentration, and will have to be determined empirically. After loading, the column can be washed with several column volumes of PBS, or other equilibration buffer, to remove small molecule contaminants, as well as DNA and lipids. The column can be eluted with PBS+500 mM additional NaCl, or a similar buffer with a pH of ˜7.2 and an ionic strength equivalent to ˜600 mM NaCl. The heparin eluate can then be processed further by affinity purification.

IMAC Polishing of Polyhistidine-Tagged Proteins

A chelating resin, such as Pharmacia Chelating Sepharose®, charged with a metal ion such as nickel, can be equilibrated in PBS. The composition of the IMAC equilibration buffer is not critical, although the pH should be 7 or greater to encourage efficient binding, and the buffer must not contain imidazole or histidine. Heparin eluate can be applied to the IMAC column, followed by several column volumes of equilibration buffer wash. Some non-tagged protein impurities typically bind to the resin, and can be washed away with equilibration buffer adjusted to 10-20 mM imidazole. The IMAC column can be eluted with PBS adjusted to 50 mM imidazole. Different tagged constructs exhibit varying strengths of IMAC binding, and in some cases imidazole concentrations greater or less than 50 mM may be required for efficient elution.

Protein-A/G Purification of Fc-Tagged Proteins

An immobilized protein-A or protein-G support, such as Protein-A HyperD, can be equilibrated in PBS or similar buffer. Cell culture supernatant can then be perfused through the column. The volumetric binding capacity of the Protein-A/G column will vary with feed target protein concentration, and will have to be determined empirically. After loading, washing with several column volumes of equilibration buffer will remove some impurities. Further impurity removal can be obtained with sub-eluting concentrations of MgCl₂, below 3 molar, for example, 2.5 molar MgCl₂ has been successfully used. MgCl₂ dissolved in water is naturally quite acidic, and wash and elution buffers can be prepared by titrating the solution pH up to neutrality with ethanolamine. After column washing, the Fc-tagged protein can be eluted with 4 molar MgCl₂.

MEP-Hypercell Purification of Fc-Tagged Proteins from Heparin Eluates

MEP-Hypercell® resin from BioSepra® (other alternative synthetic antibody-specific affinity ligands are also commercially available and may behave similarly) can be equilibrated in PBS or a similar buffer. Concentrated heparin column eluate can then be applied. The binding capacity of the Hypercell® column may vary with different Fc-tagged proteins, and will have to be determined empirically. After loading, the column can be washed with several column volumes of equilibration buffer to remove impurities. Washes with sub-eluting ethylene glycol concentrations less than 50% may provide additional impurity clearance. The MEP-Hypercell® column can be eluted with PBS adjusted to 50% weight/volume ethylene glycol.

The Examples, which follow, are illustrative of specific embodiments of the invention, and various uses thereof. They are set forth for explanatory purposes only, and are not to be taken as limiting the invention.

### EXAMPLES

Estimates of percent capture efficiencies and yields presented here are semi-quantitative, based on relative band intensities of protein spots from transblotted SDS gels probed with antibodies to the fusion tags. It would be clear to one of skill in the art that the purification steps described in the following examples can be either preformed in a column, a beaker, a flask, a test tube, or equivalent thereof.

### Example 1

Target Capture from Dilute Culture Suspension—Concentration on Immobilized Heparin

In this example, a carboxy-terminal 6×His-tagged 4-helix bundle human cytokine was expressed in a transient mammalian cell culture system with expression levels in the range of 1 mg/liter. Briefly, the DNA construct (an IL9ra covalent dimer construct) encoding the tagged-protein was made by cloning the cytokine gene into a plasmid suitable for secreted protein expression in a transient mammalian cell culture system. DNA encoding the 6×His tag was cloned in-frame with the cytokine but immediate proceeding the 3-prime end of the cytokine. An in-frame stop codon terminated the “cytokine-6×His tag gene. Attempts to capture the target molecule in a single step, by copper-IDA IMAC chromatography, gave poor results. Less than 25% of the target molecule present in the supernatant could be retrieved. When the same culture supernatant was passed over an immobilized heparin column, using the inventive method, the capture of the target molecule was nearly 100%. Washing of the heparin column removed interfering small molecules and concentrated the proteins. In a separate experiment (data not shown), the 0.5 molar salt eluate of the heparin column was then applied to a nickel-IDA IMAC column, where binding of the 6×His-tagged cytokine was again nearly quantitative. A wash with 10 mM imidazole eluted weakly bound non-tagged proteins, and the resulting 50 mM imidazole elution of the column produced 6×His-tagged cytokine at greater than 99% purity as judged by silver stained SDS gels.

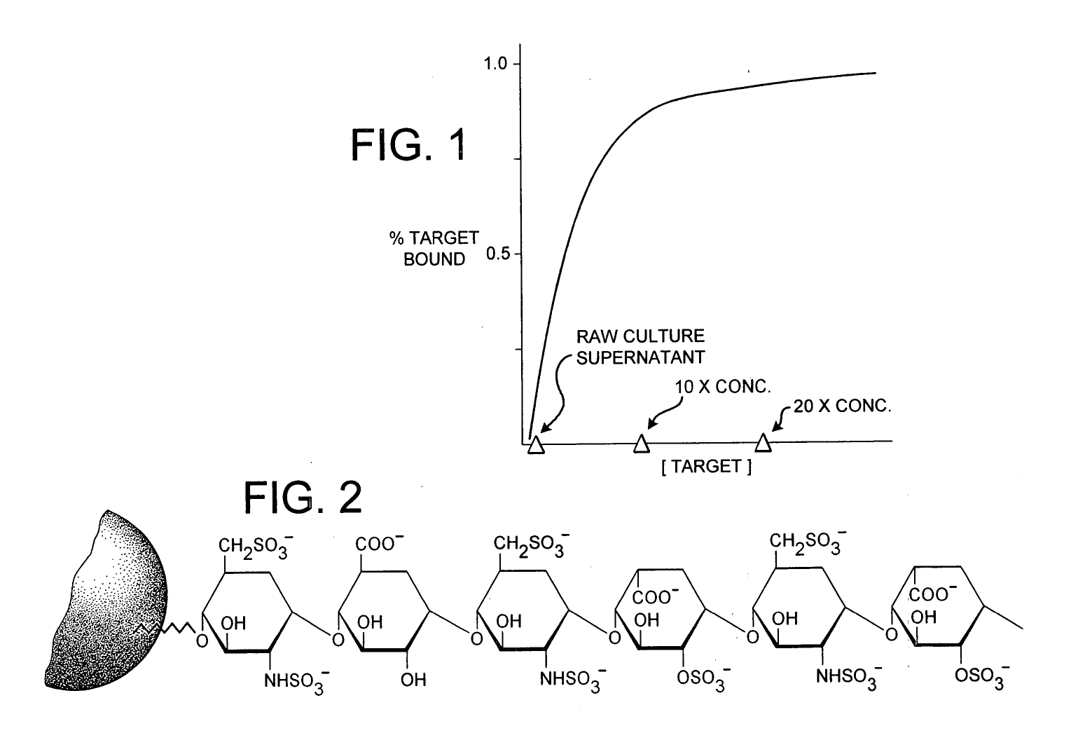

FIG. 1 presents an idealized binding isotherm illustrating the difficulty frequently encountered in capturing tagged targets, such as poly-His fusions on IMAC matrixes, from low-expression cell culture supernatants. Low target titers limit the fraction bound to the matrix, producing poor capture efficiency and elution yield. Sufficient pre-concentration of the supernatant will shift the system to a more favorable position on the isotherm, where capture efficiency will approach 100%. Concentration can be readily accomplished with membrane-based devices, but the tangential flow filtration systems required to efficiently process all but very small volumes of supernatant in a reasonable time are costly, and may be unavailable to many workers. In this invention, immobilized heparin columns were successfully used as “protein sinks” that bind most proteins from applied cell culture supernatants, including the tagged targets, producing concentrated eluates that can be further purified on affinity matrixes. The schematic diagram of an immobilized heparin chromatography resin shown in FIG. 2 explains its utility as a generic concentration medium. The repeating d-glucosamine/uronic acid disaccharide polymer carries an extremely high negative charge density of ˜2.3/residue1, binding a broad spectrum of proteins through their basic amino acids, at or near physiological ionic strength and pH.

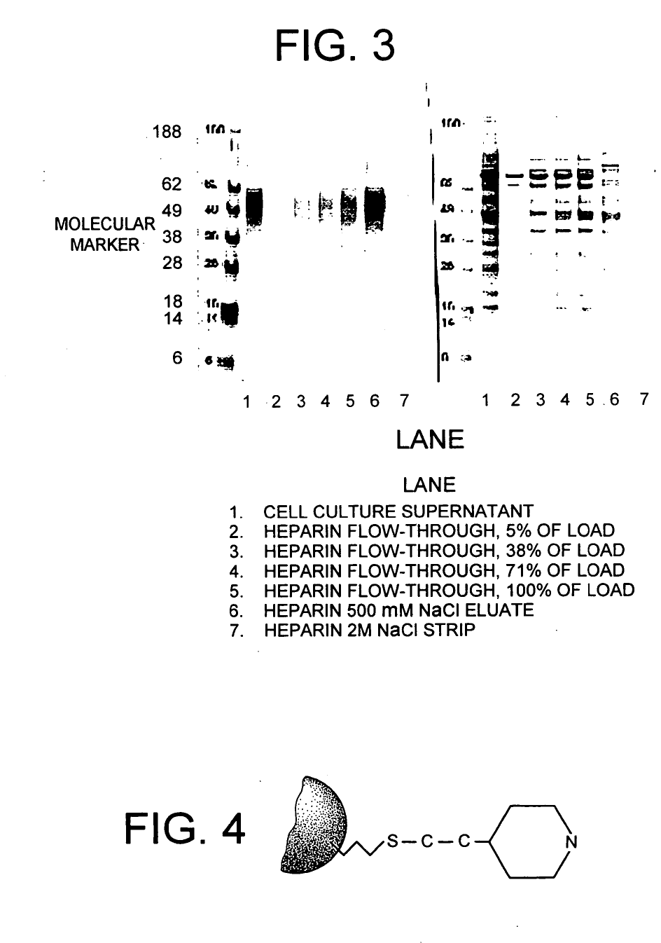

FIG. 3 shows the results of a separate experiment of loading Millipore ProsepTM Heparin with unadjusted cell culture supernatant to a target of 10 mg/ml total protein. The culture expressed a target cytokine at <1 mg/liter. The column was equilibrated in 50 mM NaPi at pH 6.5, and eluted with 500 mM NaCl in equilibration buffer. Gel/blot lanes were loaded in proportion to their pool volumes, to allow visual approximation of % yield of the target cytokine and total protein. The results show that although some target was lost to the non-binding F-T, eluate yield was >75%. In this case, a 10-fold concentration factor was obtained. Target binding no doubt was also promoted by the basic pI of the tagged cytokine. In extending this technique to other targets, lowering the ionic strength of the cell culture supernatant by dilution, and/or lowering the load pH into the 6-6.5 range to partially protonate poly-His tags, may be necessary to obtain similar performance.

### Example 2

Fc-Fusion Targets—Alternative Systems for Capture and Purification

In this Example, isolation and purification of Fc-Fusion targets are described. The Protein-A/Fc affinity binding interaction offers very efficient capture of Fc-tagged targets from cell culture supes, even at very low titers, as well as very high purification factors, typically yielding >90% purity across a single step. The in vivo half lives of smaller targets can also be markedly improved by the added molecular weight of the Fc tag. However, the classic acidic elution conditions (pH<3) used with Protein-A cause aggregation of some Fc-fusion targets, as well as loss of bioactivity. In this invention, non-acidic elution conditions were successfully employed for Protein-A as well as for the Fc-selective synthetic pseudoaffinity matrixes MEP Hypercel®, as two alternatives for the capture and purification of Fc-fusion targets.

In one example, a human IgG1 subtype Fc-tagged (amino-terminal or carboxy-terminal) 4-helix bundle human cytokine was expressed in a transient mammalian cell culture system with expression levels in the range of 1 mg/liter. Briefly, the DNA construct encoding the tagged-protein was made by cloning the cytokine gene (IL9ra) into a plasmid suitable for secreted protein expression in a transient mammalian cell culture system. DNA encoding the Fc tag was cloned in-frame with the cytokine immediate between the signal sequence of the cytokine and the cytokine for the amino-terminal Fc-tagged cytokine or immediately proceeding the 3-prime end of the cytokine for the carboxy-terminal Fc-tagged cytokine. An in-frame stop codon terminated the “Fc-tagged cytokine” genes. The Fc-tagged cytokine could be captured on immobilized Protein-A with good efficiency. It appeared that less than 25% of the cytokine remained unbound in the column flow-through. However, this cytokine was well known to lose its biological activity at pH values below 6, so classical Protein-A elution conditions were out of the question. After binding, the Protein-A column was washed with 2.5 molar MgCl₂ buffered to pH ˜7.4 with ethanolamine to elute non-specifically bound untagged proteins. A subsequent wash with 4 molar MgCl₂ buffered to pH ˜7.4 with ethanolamine eluted the Fc-tagged cytokine with an apparent yield of greater than 75%. Size exclusion chromatographic analysis of the protein showed no evidence of aggregation, and the purified cytokine showed typical biological specific activity in a cell based assay.

In another example, this same Fc-tagged cytokine was captured directly on MEP-Hypercell. In this case binding efficiency was less than 50%. When the Fc-tagged cytokine was first concentrated using the immobilized heparin technique, subsequent binding to the MEP-Hypercell was improved, to approximately 75% efficiency. The distinguishing feature of the MEP-Hypercell option is that the 50% ethylene glycol elution is very nearly quantitative. In contrast, while Protein-A capture was very good, elution efficiency was only about 75%, so the single step Protein-A method and the two step heparin/Hypercell method give roughly equivalent overall yields. This provides options for the investigator. If a Fc-tagged target molecule appears to be sensitive to high MgCl₂, for example, the low-salt glycol elution option with MEP-Hypercell may be more viable.

A schematic of the mercaptoethyl pyridine ligand of MEP HypercelTM is shown in FIG. 4. At pH values above ˜5.5 the pyridine ring is uncharged and hydrophobic, and the thioether moiety gives the ligand some of the Fc selectivity characteristic of “T-Gels”. The two intervening carbons provide spacing between the ring and the sulfur that is optimal for antibody binding, and also provides some additional hydrophobic interaction2. The default elution condition for this matrix is a pH drop to 4.0, where the pyridine and the bound Fc become protonated, and efficient elution is assured by the resulting charge repulsion. While these conditions are less stringent than Protein-A elution buffer (pH<3), pH 4 may still be damaging to some target proteins. An ongoing effect was initiated to develop mild elution conditions at near-physiologic pH values.

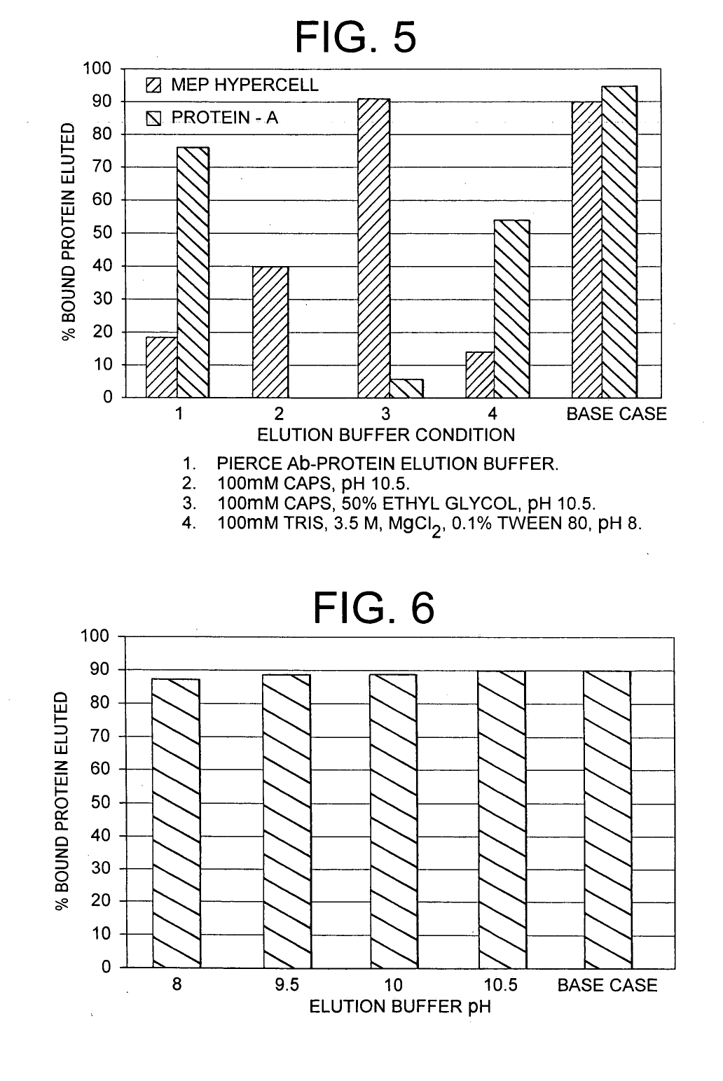

FIGS. 5 and 6 show elution yield results from a screen of alternative elution conditions for MEP Hypercell™ and rProtein-A Sepharose Fast Flow™. In all cases the columns were equilibrated in TBS and loaded to 10 mg/ml. The test protein was a Fc-tagged cytokine that had previously been purified on Protein-A by conventional means, with consequent generation of aggregates that amounted to ˜50% of the total protein by SEC HPLC. Aggregates were removed by prep-scale SEC to yield a final preparation with >80% monomer content. The test buffer blends were chosen after a scan of the literature for agents that have been found to be effective at disrupting affinity interactions. The Pierce elution buffer is a proprietary formulation, thought to contain a chaotropic salt and a mobile phase modifier such as ethylene glycol.

The results in FIG. 5 show that MEP Hypercell™ can be eluted very effectively with 50% ethylene glycol in an alkaline buffer. Alkaline pH alone, chaotropic salt, and the proprietary Pierce buffer were only nominally effective eluants. Elution of Protein-A was ˜75% efficient with the proprietary Pierce buffer, and ˜50% efficient with the 3.5 Molar MgCl₂ buffer. Non-eluting protein was subsequently recovered with the base case acidic buffer. Later tests showed that increasing the MgCl₂ concentration to 4 Molar boosted the elution yield into the 70-75% range (data not shown). Though not quantitative, this yield range is actually very favorable when one considers that a 20-30% loss of monomeric protein would be seen when prep-scale SEC is used to remove aggregates from Protein-A eluate produced by the classical acidic elution method. SEC HPLC analysis of these eluates showed no generation of additional aggregates by any of the non-acidic experimental elution buffers.

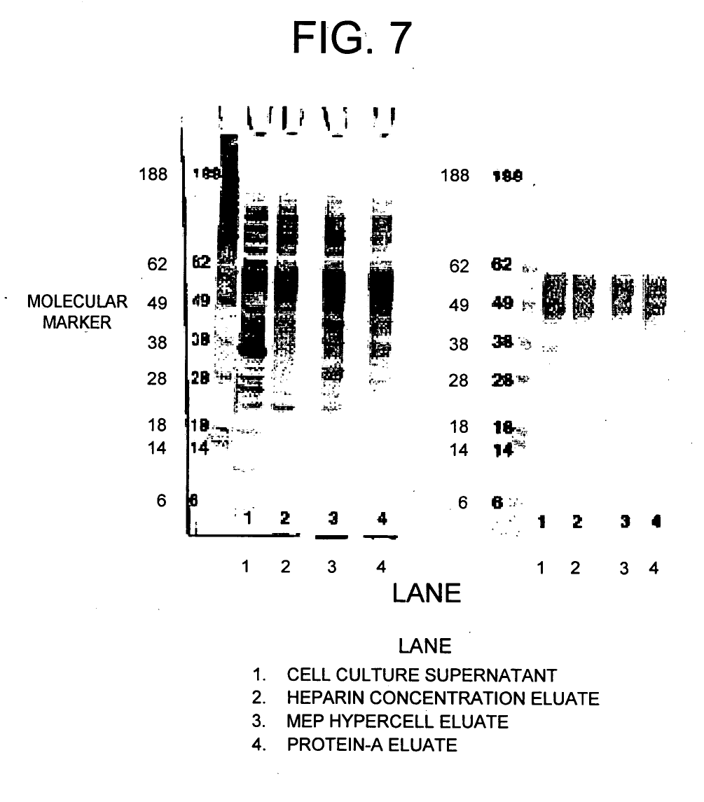

FIG. 6 shows results from an effort to determine the pH dependence of elution yield in the MEP Hypercell ethylene glycol system. Only a very nominal difference in elution yield of ˜5% was observed over the pH range of 8 to 10.5. This system makes MEP Hypercell capture and purification of Fc-tagged targets a particularly gentle and versatile method. One advantage of Protein-A over MEP Hypercell, however, is its greater capture efficiency from culture supernatants at low target titers. Feeds for MEP Hypercell must generally be concentrated prior to application, either by the Heparin method or by membrane concentration. Given a pre-concentrated feed, however, MEP Hypercell can deliver yield and purity comparable to Protein-A chromatography. FIG. 7 shows a gel and blot from an experiment in which cell culture supernatant containing an Fc-tagged target cytokine at ˜1 mg/liter was in one case pre-concentrated by immobilized heparin chromatography, and the resulting crude eluate applied to MEP Hypercell, eluted with PBS+50% ethylene glycol. In the second case, culture supe was applied directly to a Protein-A Hyper-DTM column, eluted with 3.5 Molar MgCl₂, pH 7.5. Gel and blot lanes were loaded in proportion to their respective pool volumes, to allow visual estimation of % yield. Transblot intensities imply that yield on the heparin pre-concentration step was ˜>75%, and overall, the yield from the heparin pre-conc+MEP Hypercell® protocol was approximately equal to that seen with direct loading of culture supe to Prot-A followed by neutral elution with MgCl₂. Gel purity of the MEP Hypercell eluate appears to be comparable, though not quite as clean, as the Protein-A preparation. Together, these methods provide two very viable alternatives for capture and purification of acid-sensitive Fc-tagged targets from cell culture supernatants.

### CONCLUSION

Mammalian cell expression constructs used in the early discovery and screening phases of therapeutic recombinant protein development typically secrete targets at ˜1-2 mg/liter or less. Such low expression often forces the use of purification tags such as poly-histidine or Fc fusion, but these systems introduce their own difficulties. Capture of His-tagged targets is often poor at low titers, and some targets suffer aggregation and loss of bioactivity when their Fc fusions are eluted from protein-A matrixes by conventional acidic desorption. Immobilized heparin resins are readily available, scalable, and inexpensive alternatives to TFF devices for concentration of low-titer cell culture supernatants. Mild non-acidic elution conditions were developed for two alternative affinity purification platforms for Fc fusions. Heparin pre-concentration of culture supe followed by MEP Hypercell purification offers overall yield and purity comparable to that obtained with direct loading of protein-A followed by non-acidic elution. These strategies have been valuable additions to our purification toolbox, as the use of tag systems has gained acceptance and become an approach of choice for early biological lead purification development.

It should be understood that the foregoing disclosure emphasizes certain specific embodiments of the invention and that all modifications or alternatives equivalent thereto are within the spirit and scope of the invention as set forth in the appended claims.

What is claimed:

1. A method for purifying a tagged protein from a protein preparation, comprising:
(a) concentrating the tagged protein in the protein preparation with a negatively charged capture support, comprising the steps of:
(i) contacting the protein preparation with the capture support;
(ii) washing the capture support with a capture support washing buffer of low ionic strength to remove interfering molecules but not the tagged protein from the capture support; and
(iii) eluting the tagged protein from the capture support with a capture support eluting buffer of high ionic strength;
(b) purifying the tagged protein from the eluate of step (a) (iii) with a tag-specific affinity support comprising the steps of:
(i) contacting the eluate of step (a) (iii) with the tag-specific affinity support;
(ii) washing the affinity support with affinity support washing buffer of low ionic strength to remove some impurities but not the tagged protein from the affinity support; and
(iii) eluting the tagged protein from the affinity support with an affinity support eluting buffer.

2. The method of claim 1, wherein the capture support washing buffer and the affinity support washing buffer comprise an ionic strength equivalent to about 50 mM to about 150 mM salt equivalent.

3. The method of claim 2, wherein the capture support eluting buffer comprises an ionic strength equivalent to at least about 500 mM salt equivalent.

4. The method of claim 3, wherein the capture support is applied to a column before or after contacting with the protein preparation.

5. The method of claim 3, wherein the affinity support is applied to a column before or after contacting with the eluate of the capture support.

6. The method of claim 3, wherein the negatively charged capture support comprises a polyanion immobilized on a solid support.

7. The method of claim 6, wherein the polyanion comprises one or more compounds selected from the group consisting of heparin, heparin sulfate, dextran sulfate, chondroitin sulfate, polyuronic acid, hyaluronic acid, Dermatan Sulfate, Alginic acid, and carboxymethylcellulose.

8. The method of claim 7, wherein the polyanion is heparin.

9. The method of claim 3, wherein the tagged protein is a polyhistidine-tagged protein, and wherein the affinity support comprises an immobilized metal affinity chromatography support.

10. The method of claim 9, wherein the immobilized metal affinity chromatography support comprises nickel nitrilotriacetic acid, and wherein the affinity support eluting buffer comprises at least 50 mM imidazole.

11. The method of claim 10, wherein the polyhistidine-tagged protein is a 6× histidine tagged cytokine with a four-helix bundle motif.

12. The method of claim 3, wherein the tagged protein is an Fc-tagged protein, and wherein the affinity support comprises one or more from the group consisting of protein A, protein G, and an antibody-specific affinity matrix.

13. The method of claim 12, wherein the affinity support eluting buffer is a non-denaturing buffer with a pH range between about pH 7.0 and about pH 8.0.

14. The method of claim 13, wherein the Fc-tagged protein is an Fc-tagged cytokine with a four-helix bundle motif.

15. The method of claim 13, wherein the affinity support comprises protein A and/or protein G, and wherein the affinity support eluting buffer comprises at least 4 molar MgCl₂.

16. The method of claim 13, wherein the affinity support comprises an antibody-specific affinity support, and wherein the affinity support eluting buffer comprises 50% ethylene glycol.

17. A method for purifying a polyhistidine-tagged cytokine with a four-helix bundle motif from a protein preparation, comprising:
(a) concentrating the tagged protein in the protein preparation with a negatively charged capture support, wherein the negatively charged capture support comprises heparin, comprising the steps of:
(i) contacting the protein preparation with the capture support;
(ii) washing the capture support with a capture support washing buffer of an ionic strength equivalent to a concentration of about 50 mM to about 1 M to remove interfering molecules but not the tagged protein from the capture support; and
(iii) eluting the tagged protein from the capture support with a capture support eluting buffer of an ionic strength equivalent to a concentration of about 50 mM to about 1 M;
(b) purifying the tagged protein from the eluate of step (a) (iii) with a tag-specific affinity support, wherein the affinity support comprises nickel nitrilotriacetic acid immobilized on a solid support, comprising the steps of:
(i) contacting the eluate of step (a) (iii) with the affinity support;
(ii) washing the affinity support with affinity support washing buffer of an ionic strength equivalent to a concentration of about 50 mM to about 1 M to remove some impurities but not the tagged protein from the affinity support; and
(iii) eluting the tagged protein from the affinity support with an affinity support eluting buffer comprising at least 50 mM imidazole.

18. A method for purifying an Fc-tagged protein from a protein preparation, comprising:
(a) concentrating the tagged protein in the protein preparation with a negatively charged capture support, wherein the negatively charged capture support comprises heparin, comprising the steps of:
(i) contacting the protein preparation with the capture support;
(ii) washing the capture support with a capture support washing buffer of an ionic strength equivalent to a concentration of about 50 mM to about 2 M to remove interfering molecules but not the tagged protein from the capture support; and
(iii) eluting the tagged protein from the capture support with a capture support eluting buffer of an ionic strength equivalent to a concentration of about 50 mM to about 2 M;
(c) purifying the tagged protein from the eluate of step (a) (iii) with a tag-specific affinity support, wherein the affinity support comprises protein A and/or protein G immobilized on a solid support, comprising the steps of:
(i) contacting the eluate of step (a) (iii) with the affinity support;
(ii) washing the affinity support with affinity support washing buffer of an ionic strength equivalent to a concentration of about 50 mM to about 2 M to remove some impurities but not the tagged protein from the affinity support; and
(iii) eluting the tagged protein from the affinity support with an affinity support eluting buffer, comprising at least 4 molar MgCl₂ at a pH range between about pH 7.0 and about pH 8.0.

19. A method for purifying an Fc-tagged protein from a protein preparation, comprising:
(a) concentrating the tagged protein in the protein preparation with a negatively charged capture support, wherein the negatively charged capture support comprises heparin, comprising the steps of:
(i) contacting the protein preparation with the capture support;
(ii) washing the capture support with a capture support washing buffer of an ionic strength equivalent to a concentration of about 50 mM to about 2 M to remove interfering molecules but not the tagged protein from the capture support; and
(iii) eluting the tagged protein from the capture support with a capture support eluting buffer of an ionic strength equivalent to a concentration of about 50 mM to about 2 M;
(b) purifying the tagged protein from the eluate of step (a) (iii) with a tag-specific affinity support, wherein the affinity support comprises an antibody-specific affinity support, comprising the steps of:
(i) contacting the eluate of step (a) (iii) with the affinity support;
(ii) washing the affinity support with affinity support washing buffer of an ionic strength equivalent to a concentration of about 50 mM to about 2 M to remove some impurities but not the tagged protein from the affinity support; and
(iii) eluting the tagged protein from the affinity support with an affinity support eluting buffer with a pH range between about pH 7.0 and about pH 8.0.

20. A kit for the capture and purification of a tagged protein comprising in separate containers a negatively charged capture support, and a tag-specific affinity support.

21. The kit of claim 20, wherein the capture support comprises heparin immobilized on a solid support.

22. The kit of claim 21, wherein the tagged protein is a polyhistidine-tagged protein, and wherein the affinity support comprises nickel nitrilotriacetic acid immobilized on a solid support.

23. The kit of claim 21, wherein the tagged protein is an Fc-tagged protein, and wherein the affinity support comprises protein A and/or protein G immobilized on a solid support.

24. The kit of claim 21, wherein the kit further comprises one or more of the following: a capture support washing buffer, a capture support eluting buffer, an affinity support washing buffer, an affinity support eluting buffer, and instructions for using the kit.
